(19) United States
(12) Patent Application Publication
(10) Pub. No.: US 2022/0254513 A1
(43) Pub. Date: Aug. 11, 2022

(54) INCIDENCE RATE MONITORING METHOD, APPARATUS AND DEVICE, AND STORAGE MEDIUM

(71) Applicant: PING AN TECHNOLOGY (SHENZHEN) CO., LTD., Shenzhen (CN)

(72) Inventors: Xianxian CHEN, Shenzhen (CN); Xiaowen RUAN, Shenzhen (CN); Liang XU, Shenzhen (CN)

(73) Assignee: PING AN TECHNOLOGY (SHENZHEN) CO., LTD., Shenzhen (CN)

(21) Appl. No.: 17/617,293

(22) PCT Filed: Jun. 30, 2020
(86) PCT No.: PCT/CN2020/099450

§ 371 Date: Dec. 7, 2021

(30) Foreign Application Priority Data
Aug. 1, 2019 (CN) 2019 10706318.4

(51) Int. Cl. G16H 70/60

(52) U.S. Cl. CPC G16H 70/60

(57) ABSTRACT

An incidence rate monitoring method, apparatus and device based on historical disease information, and a computer-readable storage medium, wherein the incidence rate monitoring method based on historical disease information includes: forming a prediction model of incidence rate monitoring based on historical disease information through continuous and autonomous learning of historical medical record data based on a combination of a preset gated recurrent neural network and an ensemble learning algorithm, and then inputting disease data based on the to-be-predicted disease into the prediction model for prediction and monitoring. The prediction model is formed by capturing a certain pattern from the historical medical record data through the combination of the above-mentioned algorithm and neural network.

### CROSS REFERENCE TO THE RELATED APPLICATIONS

This application is the national phase entry of International Application No. PCT/CN2020/099450, filed on Jun. 30, 2020, which is based upon and claims priority to Chinese Patent Application No. 201910706318.4, filed on Aug. 1, 2019, the entire contents of which are incorporated herein by reference.

### TECHNICAL FIELD

The present application relates to the field of neural network technologies, and in particular, to an incidence rate monitoring method, apparatus and device based on historical disease information, and a storage medium.

### BACKGROUND

As the integration of science and technology with economy and life is accelerating, economic and communication activities are growing, and the population flow has become increasingly frequent, which provides a favorable environment for the spread and outbreak of diseases, making the public health problems become increasingly severe. At the same time, social and natural environments are also confronted with changes. The increase in environmental pollution, natural disasters and other incidents that affect public health has also increased the possibility of public health emergencies.

How to identify a disease outbreak at an early stage, give early warnings in time and take corresponding control measures as early as possible, so as to minimize damage caused by the disease outbreak, is one of the focuses of current medical science and technology.

Such an identification method is necessary, especially in monitoring of influenza diseases, such as dengue fever, which is mainly prevalent in tropical and subtropical regions and relatively prevalent mainly in southern cities in China as one of diseases with seasonal epidemicity. This disease is affected by many prorogation and influencing factors, and its harm degree and influence are less obvious. Currently, to prevent this type of virus, the medical community mainly determines whether the disease occurs based on seasonal climate and weather and machine learning. For prediction of the incidence rate, a conventional control method is to collect samples and inducing factors in a certain region, train and test a model based on the samples and the inducing factors, and then perform disease prediction based on the model and real-time data. This method cannot effectively integrate the factors that affect the onset of the disease into one model, causing the machine to fail to learn in time, and further affecting accuracy of disease prediction.

### SUMMARY

A main objective of the present application is to provide an incidence rate monitoring method, apparatus and device based on historical disease information, and a storage medium, so as to resolve the technical problem in the prior art that accuracy of disease incidence rate monitoring through machine learning is not high.

To achieve the above-mentioned objective, according to a first aspect of the present application, an incidence rate monitoring method based on historical disease information is provided, including: obtaining historical medical record data of a disease, and classifying the historical medical record data based on different pre-formed age ranges; performing, based on the classified historical medical record data, an autonomous learning operation of model training on historical medical record data in each age range through a preset gated recurrent neural network and an ensemble learning algorithm to generate a prediction model, where the prediction model is used to predict and calculate an incidence rate of the to-be-predicted disease; and obtaining a type of the to-be-predicted disease, a to-be-predicted time point and related data before the time point, inputting the related data into the prediction model, and calculating a predicted result of the incidence rate of the to-be-predicted disease at the time point, where the related data includes case data monitored before the time point.

According to a second aspect of the present application, an incidence rate monitoring device based on historical disease information is provided, including a memory, a processor and computer-readable instructions stored in the memory and executable on the processor, where the processor implements the following steps when executing the computer-readable instructions: obtaining historical medical record data of a disease, and classifying the historical medical record data based on different pre-formed age ranges; performing, based on the classified historical medical record data, an autonomous learning operation of model training on historical medical record data in each age range through a preset gated recurrent neural network and an ensemble learning algorithm to generate a prediction model, where the prediction model is used to predict and calculate an incidence rate of the to-be-predicted disease; and obtaining a type of the to-be-predicted disease, a to-be-predicted time point and related data before the time point, inputting the related data into the prediction model, and calculating a predicted result of the incidence rate of the to-be-predicted disease at the time point, where the related data includes case data monitored before the time point.

According to a third aspect of the present application, a computer-readable storage medium is provided, where the computer-readable storage medium stores computer instructions, and when the computer instructions are run on a computer, the computer is enabled to perform the following steps: obtaining historical medical record data of a disease, and classifying the historical medical record data based on different pre-formed age ranges; performing, based on the classified historical medical record data, an autonomous learning operation of model training on historical medical record data in each age range through a preset gated recurrent neural network and an ensemble learning algorithm to generate a prediction model, where the prediction model is used to predict and calculate an incidence rate of the to-be-predicted disease; and obtaining a type of the to-be-predicted disease, a to-be-predicted time point and related data before the time point, inputting the related data into the prediction model, and calculating a predicted result of the incidence rate of the to-be-predicted disease at the time point, where the related data includes case data monitored before the time point.

According to a fourth aspect of the present application, an incidence rate monitoring apparatus based on historical disease information is provided, including: a first data obtaining module, configured to obtain historical medical record data of a disease, and classify the historical medical record data based on different pre-formed age ranges; a model training module, configured to perform, based on the classified historical medical record data, an autonomous learning operation of model training on historical medical record data in each age range through a preset gated recurrent neural network and an ensemble learning algorithm to generate a prediction model, where the prediction model is used to predict and calculate an incidence rate of the to-be-predicted disease; and an incidence prediction module, configured to obtain a type of the to-be-predicted disease, a to-be-predicted time point and related data before the time point, input the related data into the prediction model, and calculate a predicted result of the incidence rate of the to-be-predicted disease at the time point, where the related data includes case data monitored before the time point.

In the technical solution according to the present application, a prediction model of incidence rate monitoring based on historical disease information is formed through continuous and autonomous learning of historical medical record data based on a combination of a gate recurrent unit (GRU) in a preset gated recurrent neural network and an ensemble learning algorithm. The prediction model is formed by capturing certain patterns from the historical medical record data through the combination of the algorithm and the neural network. The combination of the GRU network and the ensemble learning algorithm not only reduces a data memory amount of the model, but also improves efficiency of disease prediction, thereby enabling rapid and accurate prediction of disease prevalence, implementing timely start of early warnings, and facilitating prevention and control over epidemic diseases by relevant staff.

### BRIEF DESCRIPTION OF THE DRAWINGS

FIG. 1 is a schematic flowchart of Embodiment 1 of an incidence rate monitoring method based on historical disease information according to the present application;

FIG. 2 is a schematic flowchart of Embodiment 2 of an incidence rate monitoring method based on historical disease information according to the present application;

FIG. 3 is a schematic structural diagram of a server running environment related to a solution according to an embodiment of the present application; and

FIG. 4 is a schematic diagram of function modules in an embodiment of an incidence rate monitoring apparatus based on historical disease information according to the present application.

### DETAILED DESCRIPTION OF THE EMBODIMENTS

Embodiments of the present application provide an incidence rate monitoring method, apparatus and device based on historical disease information, and a storage medium. The incidence rate monitoring method based on historical disease information is implemented by combining an algorithm and a neural network. Through the combination of a GRU as the neural network and a random forest algorithm, a corresponding prediction model is generated through long-time learning and training of medical records, and patterns, commonalities and effectiveness of disease onset can be fully captured, which improves statistical accuracy of the data model. The number of patents is predicted based on the constructed prediction model. Because of a learning manner of the GRU, a data information memory time of the model is prolonged, and memorized information is relatively simplified, thus implementing the prediction for a longer time. Compared with a conventional model prediction manner, the present solution has higher accuracy, which facilitates disease prevention and control by medical staff.

To enable a person skilled in the art to better understand the solution of the present application, the embodiments of the present application are described below with reference to the accompanying drawings in the embodiments of the present application.

Terms “first”, “second”, “third”, “fourth”, etc. (if any) in the specification, claims, and accompanying drawings of the present application are used to distinguish between similar objects without having to describe a specific order or sequence. It should be understood that data used in this way may be interchanged under appropriate circumstances, so that the embodiments described herein can be implemented in an order other than that illustrated or described herein. In addition, the term “including” or “having” and any variants thereof are intended to cover non-exclusive inclusions. For example, a process, method, system, product, or device that includes a series of steps or units is not necessarily limited to those steps or units clearly listed, and may include other steps or units that are not clearly listed or are inherent to the process, method, product, or device.

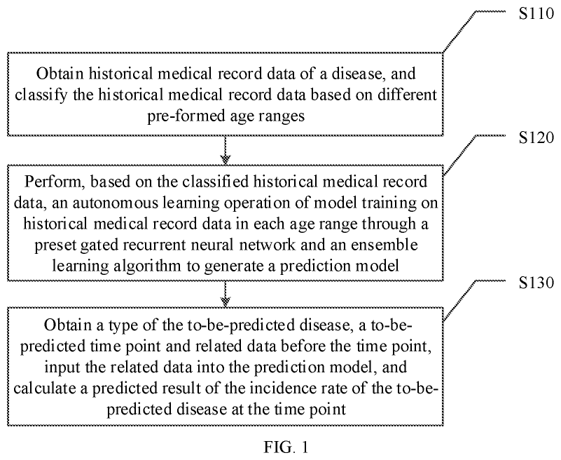

For ease of understanding, a specific process of an embodiment of the present application is described below. Referring to FIG. 1, FIG. 1 is a flowchart of an incidence rate monitoring method based on historical disease information according to an embodiment of the present application. In this embodiment, the incidence rate monitoring method based on historical disease information specifically includes the following steps.

Step S110: Obtain historical medical record data of a disease, and classify the historical medical record data based on different pre-formed age ranges.

In this step, historical medical record data of dengue fever may be retrieved from a medical record database of a current open medical system, or obtained by extracting samples from some medical expert consultation sites on the Internet.

Specifically, the historical medical record data may be specifically extracted based on conditions such as a time, a region and a medical record type. For example, medical records covering regions A, B and C and several months with the highest number of patients in a certain year need to be selected, and it is further necessary to give priority to medical records covering all risk levels among the medical records obtained in the several months. Such practice can ensure comprehensiveness of the obtained historical medical record data.

In practice, the data may be obtained from a network of a disease monitoring center in a preset region. Optionally, the disease monitoring center may be a medical institution, a school, a childcare institution, a pharmacy or the like. These monitoring centers separately perform disease monitoring and data collection on corresponding target population. A place that meets preset conditions may be selected as a source for data acquisition. The preset conditions may include the number of people, a scale, or even proportional extraction from all monitoring points, or the like. For example, a school and a childcare institution with a preset number of students are selected as acquisition points. For another example, a pharmacy reaching a preset scale (for example, on the basis of daily turnover) is selected as an acquisition point. For another example, a hospital reaching a preset scale (for example, on the basis of the daily number of patients) is selected as an acquisition point.

In this embodiment, the medical record data includes information about a patient and a disease type, such as age, gender, occupation and residence. Preferably, to make the data available for reference, a longer historical time is set for data selection, and an option is a time period of 2 or 3 years that is close to the current time point. Data selected in this way is available for more real-time reference, which can avoid special mutation of some viruses.

In this embodiment, the historical medical record data may be classified based on crowds or disease onset features. In practice, because different people have different lifestyles or habits, different living habits may also lead to changes in the incidence rate of dengue fever. For example, people may be classified into a high-density living crowd, a factory crowd, a high-tech occupational crowd, etc. Because the high-density living crowd lives in an environment with relatively poor hygiene conditions, more mosquitoes may be attracted, where dengue fever is spread through mosquitoes.

Moreover, the patients may also be classified based on disease severity in historical medical records. For example, they may be classified into a typical dengue fever type, a mild dengue fever type and a severe dengue fever type, and the number of patients in each type is counted.

In practice, when this method is used to predict the number of cases, it is usually targeted on prediction of a certain disease, but a case without disease types set is not ruled out. After the historical medical record data is obtained, it is further necessary to introduce classification of disease types in the classification process in addition to the above-mentioned classification dimensions. Specifically, the diseases herein should be understood as diseases with spreading and infection characteristics, such as dengue fever, influenza, hand-foot-mouth disease, measles, mumps and other epidemic diseases.

Step S120: Perform, based on the classified historical medical record data, an autonomous learning operation of model training on historical medical record data in each age range through a preset gated recurrent neural network and an ensemble learning algorithm to generate a prediction model, where the prediction model is used to predict and calculate an incidence rate of the to-be-predicted disease.

In this step, a GRU is a recurrent neural network, which has the potential to learn a long observation sequence. In this solution, the GRU is used as a main way to construct a training model, and the ensemble learning algorithm is used to control and train a variety of different data to construct the model in the GRU network, so that it is not required to train a plurality of models separately for disease prediction. Moreover, the model constructed through the GRU may be called a GRU model. Specifically, some gates are constructed to store information, and a gradient does not disappear quickly during the model training process. In addition, the model built in this way does not need to memorize much information, and its duration for storage is much longer than that of other models.

Step S130: Obtain a type of the to-be-predicted disease, a to-be-predicted time point and related data before the time point, input the related data into the prediction model, and calculate a predicted result of the incidence rate of the to-be-predicted disease at the time point, where the related data includes case data monitored before the time point.

In this embodiment, to predict the number of patients with a certain disease in a period of time in the future through the above-mentioned steps, it is necessary to determine a time period for prediction, and it is also necessary to perform the prediction by combining medical record data at a certain time point relatively close to the current time period. The selected medical record data herein may be medical record data that overlaps the historical medical record data in step S110, or certainly may be medical record data that does not overlap.

To further improve accuracy of prediction, after the historical medical record data is obtained, step S110 of the solution may further include analyzing commonalities/onset patterns of the historical medical record data. The commonalities or pattern analysis herein refers to analyzing the onset patterns in the historical medical record data, such as collecting statistics on living environments of all patients and comparing them with each other, so as to determine whether the living environment is one of the causes of the epidemic disease and whether it is a factor that leads to the increase or decrease in the number of patents in the year. For another example, whether a virus has mutated needs to be determined. If yes, it is necessary to combine the mutation with the environment for further analysis, so as to determine whether there is a relationship between the mutation of the virus and the environment, etc. Information obtained in the analysis may be integrated into the model through the model training in step S120 by using the ensemble learning algorithm, which can ensure accurate prediction of the number of patients.

In this embodiment, further, after the historical medical record data is classified, it is also possible to perform a single analysis on each category after the classification, and analyze different categories separately. The analysis process includes collecting statistics on the number of patients and statistics on disease onset factors, etc. That is, during model training, one model may be trained for each category to be used alone.

For example, the obtained historical medical record data is medical records in region A for three consecutive years before the current moment, the medical record data in the three years is classified on a yearly basis first, then the medical records of the patients suffering from the disease in each year are classified based on three categories: typical dengue fever, mild dengue fever and severe dengue fever, and then changes in the number of patients in each category in each year are compared.

In addition, after the historical medical records are classified, external factors of disease onset are also analyzed, such as how the external environment was in the time when dengue fever occurred. Various data in the three years is compared successively to finally output the onset patterns. These patterns are also stored as medical record data, and they are trained together during model training. After being processed in this way, the data is trained into the model, which makes the model more comprehensive. During the prediction, more data can be combined for analysis and prediction, which further improves prediction accuracy and also increases the intensity and pertinence of the prevention and control over these diseases.

Further, in this embodiment, the step of performing, based on the classified historical medical record data, an autonomous learning operation of model training on historical medical record data in each age range through a preset gated recurrent neural network (GRU) and an ensemble learning algorithm to generate a prediction model includes:

extracting at least two training samples from classified historical medical record data of each category through random sample extraction;

selecting one training sample from the extracted training samples as an initial sample, and performing preliminary model training based on the initial sample to obtain a model prototype of the prediction model; and

adding an information storage gate to the model prototype through the gated recurrent neural network, and using the training samples extracted from each category to perform secondary deep ensemble learning training on the model prototype with the added information storage gate by using the ensemble learning algorithm, so as to construct the prediction model.

In this implementation process, after the model is created based on the GRU neural network, the subsequent training and integration of the model based on the medical record data may specifically include:

first using a Bootstraping method to randomly select M samples from the historical medical record data obtained in step S110, and performing sampling for n_tree times in total to generate n_tree training samples to form a training set;

training n_tree decision tree models based on the created training model for n_tree training sets;

on the assumption that there are n training sample features for a single decision tree model, selecting an optimal feature for each split based on an information gain/information gain ratio/Gini index;

keeping splitting each tree model in this way, until all training samples of a node belong to the same category, where the model does not need to be pruned during the splitting training process; and

integrating a plurality of generated decision trees by using the ensemble learning algorithm to form the disease prediction model.

Further, the model trained through the combination of the GRU neural network and the ensemble learning algorithm further has the function of a regression model, and can validate regression of data to a certain extent, thereby preventing gradient dispersion of data from affecting the predicted result.

In this embodiment, the step of using the training samples extracted from each category to perform deep ensemble learning training on the model prototype with the added information storage gate by using the ensemble learning algorithm, so as to construct the prediction model may specifically further include:

performing feature splitting training on each of the training samples based on the ensemble learning algorithm to obtain first training features; and

sequentially inputting the first training features into the model prototype for deep feature training to obtain a decision tree model with multiple branches, and using the decision tree model as the prediction model.

That is, the first training features are obtained by splitting a training feature of each training sample by using the ensemble learning algorithm.

Then, the first training features are used to separately train an initial model to obtain a decision tree model with multiple branches, and the decision tree model is used as the disease prediction model.

In practice, the ensemble learning algorithm may be specifically implemented using a random forest algorithm. The algorithm has extremely high accuracy for data integration processing, and can introduce randomness, which makes a random forest not easy to be over-fitted. Moreover, the random forest also has a good anti-noise capability, and can handle high-dimensional data without feature selection. The algorithm can process both discrete data and continuous data. A data set does not need to be standardized, a training speed is fast, and a variable importance order can be obtained. More importantly, it is easy to implement parallel processing of different influencing factors.

In this embodiment, the incidence rate monitoring method based on historical disease information further includes:

obtaining medical ecological information corresponding to the historical medical record data, where the medical ecological information includes at least one of weather data, medical level data and disease monitoring data.

In practice, this step may be implemented before the related data before the time point is obtained, or may be performed at the same time when the historical medical record data is obtained from a medical system or a webpage. That is, the medical ecological information obtained in this step corresponds to the initially obtained historical medical record data, so that more change factors are introduced when the historical medical record data is used to train the prediction model, and accuracy of the prediction model can be greatly improved.

In this case, the step of training the prediction model further includes:

performing feature decomposition training on the medical ecological information by using the ensemble learning algorithm to obtain a second training feature; and

inputting the second training feature into the decision tree model for tertiary deep training learning to construct the complete prediction model.

In practice, adding the obtained medical ecological information to the training process of the model may be implemented by adding the obtained medical ecological information to the decision tree model in the above-mentioned manner and performing deep training, or by directly adding the obtained medical ecological information in the first deep training.

In this embodiment, the weather data includes an air temperature, humidity, etc. In practice, the medical ecological information may also include a crowd density, etc. During training of the disease prediction model, in the process of learning and training the model based on the data and forming the completed training model that combines the neural network (GRU) and the random forest algorithm, a stable and consolidated model is formed through continuous learning of historical medical record data via the recurrent neural network. With regard to additional training of the medical ecological information, the weather data, the medical level data, the disease monitoring data and people's health level can be used to accurately predict an disease onset probability and the total number of patients in a certain region by using the addition mechanism, and the disease onset probability and the total number of patients may be added to the model for training, so that the trained model has better comprehensiveness and higher prediction accuracy.

In this embodiment, the disease monitoring data may specifically be purchase and use data of preventive drugs in the daily life of a user, a history of consultation on physical conditions at ordinary times, etc., all of which can be used as elements to determine the user's physical health status at the current time point, and the physical health status is one of factors affecting immunity against some epidemic diseases and determining whether the diseases occur.

In this embodiment, after the step of performing, based on the classified historical medical record data, an autonomous learning operation of model training on historical medical record data in each age range through a preset gated recurrent neural network and an ensemble learning algorithm to generate a prediction model, the method further includes:

randomly capturing medical record data of a time period from the historical medical record data, and inputting the same into the prediction model to obtain a predicted value of the number of cases corresponding to the medical record data of the time period;

determining whether the predicted value meets actual incidence data corresponding to the medical record data of the time period to obtain a model verification result; and

determining, based on the model verification result, whether to perform quaternary deep training to optimize the prediction model, where the quaternary deep training is a process of repeating the secondary deep training and the tertiary deep training learning.

In practice, specifically, partial medical record data may be extracted from the historical medical record data, and input into the disease prediction model to obtain a predicted value of the number of cases in a time period corresponding to the partial medical record data;

it is determined whether the predicted value meets actual incidence data in the time period corresponding to the partial medical record data; and

based on a determining result, it is determined whether deep training is needed to optimize the disease prediction model.

The validation process may be specifically implemented by the following example.

Sequence data in a certain time period for training the disease prediction model is captured from the historical medical record data; data required by the training model corresponding to each time point is obtained from the captured sequence data to construct a training set with a preset dimension, and the training sets corresponding to the time points are sequentially input into the disease prediction model based on the time sequence, so as to train the disease prediction model. Sequence data in a certain time period for training the disease prediction model is captured from the historical medical record data; data required by the training model corresponding to each time point is obtained from the captured sequence data to construct a validation set with a preset dimension, and the validation sets corresponding to the time points are sequentially input into the disease prediction model based on the time sequence, so as to validate the multilayer GRU model.

Further, if it is determined that the model verification result is that the predicted value does not meet the actual incidence data, after the step of obtaining a type of the to-be-predicted disease, a to-be-predicted time point and related data before the time point, inputting the related data into the prediction model, and calculating a predicted result of the incidence rate of the to-be-predicted disease at the time point, the method further includes:

extracting N pieces of sample data from the historical medical record data, using an addition mechanism to update and/or reset the training samples used to train the prediction model, and training the prediction model based on the updated and/or reset training samples, where N is greater than or equal to 2.

Specifically, quantitative historical medical record data is extracted; the addition mechanism is used to update and/or reset the data for training the disease prediction model, and the disease prediction model is trained based on the updated and/or reset historical medical record data.

In this embodiment, the training for model learning is not only the learning and training of the historical medical record data, but further includes learning and updating of real-time patient data. That is, during model learning and training through the GRU, learning and training may be increased to update and improve the model. Moreover, some algorithms may be further used to tighten up the data during the learning of medical record data. For example, in addition to an RNN structure, an addition mechanism is added during propagation from t to t−1 to prevent data gradient dispersion. The update and reset functions can directly and quickly control information, and reduce and refine parameters of the data, so as to implement long-term memory of the information with fewer parameters, and provide better predictions of the number of patients.

In this embodiment, in addition to the above-mentioned learning and training, the tree model Random Forest with very high stability in machine learning may be further combined for integration, and features of historical medical record data obtained after importance screening by using Random Forest are input into the GRU for model integration, so that a more accurate prediction model can be obtained.

In this embodiment, with regard to the implementation of step 130, after the prediction model is obtained, the number of patients can be automatically predicted by obtaining and inputting to-be-predicted data into the prediction model, and the to-be-predicted data includes a prediction time point and some other experimental data. Preferably, in this implementation, the experimental data is weather data and a medical level, and historical medical record data at a time point the same as this prediction time point is extracted from the historical medical record data. For example, if the time point is March 2018, historical medical record data on March 2017, March 2016, etc. should be extracted, that is, the historical medical record data is extracted only on a month basis.

The experimental data is input into the prediction model to obtain predicted data corresponding to the number of patients at this time point.

In conclusion, in the incidence rate monitoring method based on historical disease information according to the embodiments of the present application, in the combination of the recurrent neural network and the random forest algorithm, the tree model and the recurrent neural network are integrated to improve the memory of the model on patterns of historical medical record data, and improve accuracy of the model through continuous model learning and updating. This ensures that when the model is used to predict the number of patients, the number of patients in a long time period in the future can be accurately predicted; and in addition, efficiency and speed of prediction are improved, and early epidemic warnings can be provided, having great significance in positioning and promoting the prevention and control work.

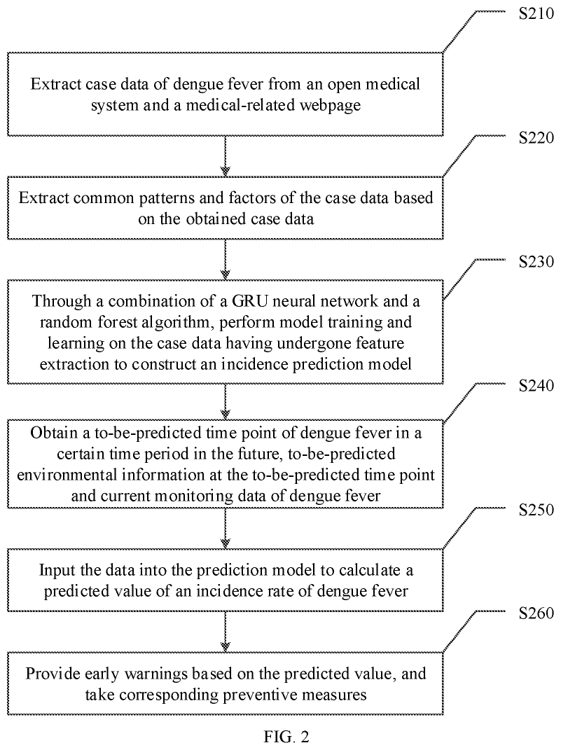

The incidence rate monitoring method based on historical disease information according to the present application is described in detail below by taking specific disease monitoring as an example. FIG. 2 shows a flowchart of specific implementation of the incidence rate monitoring method based on historical disease information, for example, prediction of dengue fever disease. The incidence rate monitoring method based on historical disease information specifically includes the following steps.

Step S210: Extract case data of dengue fever from an open medical system and a medical-related webpage.

In this step, the extracted case data includes user information, a cause of disease onset, environmental information at the time of disease onset, a medical level at that time, and other data.

Certainly, for the implementation of the step, in addition to being obtained from the system and the webpage, the data may also be obtained through a platform for some community research activities, or through investigation and statistics collection on different living crowds. In practice, preferably, data obtained from a medical station for people with different living environments is optimal, and the environment and people's living habits are relatively important factors that lead to high incidence of diseases. Obtaining data based on these factors can better reflect the incidence prediction.

Step S220: Extract common patterns and factors of the case data based on the obtained case data.

In this step, the common patterns and factors may be specifically extracted by using a conventional feature extraction algorithm, such as a keyword extraction algorithm.

Step S230: Through a combination of a GRU neural network and a random forest algorithm, perform model training and learning on the case data having undergone feature extraction to construct an incidence prediction model.

In practice, several pieces of representative case data are extracted in a random sampling manner from the case data having undergone feature extraction as training samples of the model;

one training sample is selected from the extracted training samples as an initial sample, and preliminary model training is performed based on the initial sample to obtain a model prototype of the prediction model; and an information storage gate is added to the model prototype through the GRU neural network, and the extracted training samples are used to perform deep ensemble learning training on the model prototype with the added information storage gate by using the random forest algorithm, so as to construct the prediction model.

Step S240: Obtain a to-be-predicted time point of dengue fever in a certain time period in the future, to-be-predicted environmental information at the to-be-predicted time point and current monitoring data of dengue fever.

Step S250: Input the data into the prediction model to calculate a predicted value of an incidence rate of dengue fever.

Step S260: Provide early warnings based on the predicted value, and take corresponding preventive measures.

In this embodiment, the neural network and the random forest algorithm are used for autonomous training and learning, so as to obtain patterns or commonalities of each onset through statistics collection, and predict the incidence rate in a period of time in the future based on the patterns or the commonalities. In addition to statistics collection implemented through autonomous training and learning by using the neural network and the random forest algorithm, some models are further combined to increase the concentration of statistics, for example, a tree model or an addition mechanism is used for simple memory of information, so as to improve efficiency of creating the neural network model and accuracy of prediction.

To resolve the above-mentioned problems, the present application further provides an incidence rate monitoring device based on historical disease information, which can be used to implement the incidence rate monitoring method based on historical disease information according to the embodiments of the present application. The incidence rate monitoring device based on historical disease information is physically implemented in the form of a server. Specific hardware implementation of the server is shown in FIG. 1.

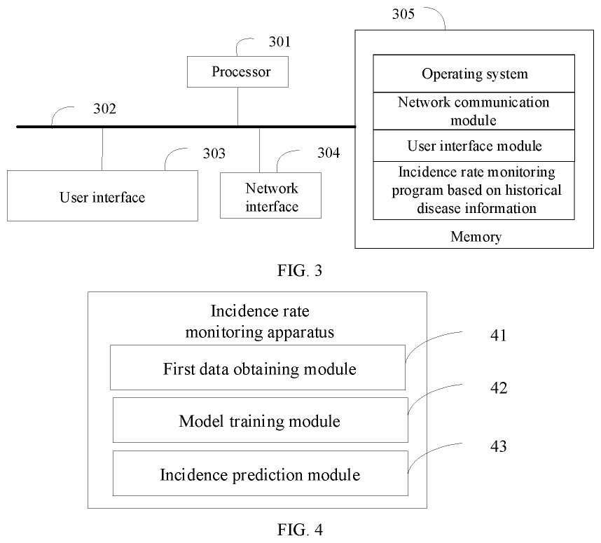

Referring to FIG. 3, the server includes a processor 301 such as a CPU, a communications bus 302, a user interface 303, a network interface 304, and a memory 305. The communications bus 302 is configured to implement connections and communication between these components. The user interface 303 may include a display and an input unit such as a keyboard. The network interface 304 may optionally include a standard wired interface and a wireless interface (such as a Wi-Fi interface). The memory 305 may be a high-speed RAM, or a stable memory (non-volatile memory), such as a magnetic disk memory. The memory 305 may optionally be a storage apparatus independent of the processor 301.

It can be understood by a person skilled in the art that a hardware structure of the device shown in FIG. 3 does not constitute a limitation to an incidence rate monitoring apparatus based on historical disease information, and may include more or fewer components than those shown, or combine some components, or have different component arrangements.

As shown in FIG. 3, the memory 305 as a computer-readable storage medium may include an operating system, a network communication module, a user interface module and an incidence rate monitoring program based on historical disease information. The operating system is a program that manages the incidence rate monitoring apparatus based on historical disease information and software resources, and supports the operation of the incidence rate monitoring program based on historical disease information and other software and/or programs.

In the hardware structure of the server shown in FIG. 3, the network interface 304 is mainly configured to access a network; the user interface 303 is configured to access case information executed on the device and data generated during the execution of a case; and the processor 301 may be configured to revoke the incidence rate monitoring program based on historical disease information stored in the memory 305, and perform operations of the following embodiments of the incidence rate monitoring method based on historical disease information.

In the embodiment of the present application, FIG. 3 may also be implemented through a mobile terminal that can be operated by touch, such as a mobile phone. A processor of the mobile terminal analyzes historical medical record data by reading program code that is stored in a buffer or storage unit for implementing the incidence rate monitoring method based on historical disease information, and performs autonomous training and learning to generate a prediction model for incidence rate monitoring based on historical disease information. In the learning process, a random forest algorithm is combined to randomly insert influencing factors that may affect disease onset to improve training accuracy of the model.

To resolve the above-mentioned problems, the present application further provides an incidence rate monitoring apparatus based on historical disease information. Referring to FIG. 4, FIG. 4 is a schematic diagram of function modules of an incidence rate monitoring apparatus based on historical disease information according to an embodiment of the present application. In this embodiment, the apparatus includes:

a first data obtaining module 41, configured to obtain historical medical record data of a disease, and classify the historical medical record data based on different pre-formed age ranges;

a model training module 42, configured to perform, based on the classified historical medical record data, an autonomous learning operation of model training on historical medical record data in each age range through a preset gated recurrent neural network and an ensemble learning algorithm to generate a prediction model, where the prediction model is used to predict and calculate an incidence rate of the to-be-predicted disease; and

an incidence prediction module 43, configured to obtain a type of the to-be-predicted disease, a to-be-predicted time point and related data before the time point, input the related data into the prediction model, and calculate a predicted result of the incidence rate of the to-be-predicted disease at the time point, where the related data includes case data monitored before the time point.

The embodiment content of the incidence rate monitoring apparatus based on historical disease information is the same as that of the incidence rate monitoring method based on historical disease information according to the embodiments of the present application, and details are not repeated in this embodiment.

In this embodiment, through the combination of a GRU as the neural network and a random forest algorithm, a corresponding prediction model is generated through long-time learning and training of medical records, and patterns, commonalities and effectiveness of disease onset can be fully captured, which improves statistical accuracy of the data model. The number of patents is predicted based on the constructed prediction model. Because of the learning manner of the GRU, a data information memory time of the model is prolonged, and memorized information is relatively simplified, thus implementing the prediction for a longer time. Compared with a conventional model prediction manner, the present solution has higher accuracy, which facilitates disease prevention and control by medical staff.

The present application further provides an incidence rate monitoring device based on historical disease information, including: a memory and at least one processor, where the memory stores instructions, and the memory and the at least one processor are interconnected by a line; and the at least one processor invokes the instructions in the memory to enable an intelligent path planning device to perform the steps of the incidence rate monitoring method based on historical disease information.

The present application further provides a computer-readable storage medium. The computer-readable storage medium may be a non-volatile computer-readable storage medium or a volatile computer-readable storage medium. The computer-readable storage medium stores computer instructions, and when the computer instructions are run on a computer, the computer is enabled to perform the following steps:

obtaining historical medical record data of a disease, and classifying the historical medical record data based on different pre-formed age ranges;

performing, based on the classified historical medical record data, an autonomous learning operation of model training on historical medical record data in each age range through a preset gated recurrent neural network and an ensemble learning algorithm to generate a prediction model, where the prediction model is used to predict and calculate an incidence rate of the to-be-predicted disease; and obtaining a type of the to-be-predicted disease, a to-be-predicted time point and related data before the time point, inputting the related data into the prediction model, and calculating a predicted result of the incidence rate of the to-be-predicted disease at the time point, where the related data includes case data monitored before the time point.

A person skilled in the art can clearly understand that for ease and brevity of description, for specific working processes of the system, apparatus and units described above, reference may be made to the corresponding processes in the foregoing method embodiments. Details are not repeated herein.

In several embodiments according to the present application, it should be understood that the disclosed system, apparatus and method may be implemented in other ways. For example, the above-described apparatus embodiments are only schematic. For example, the division of the units is merely a logical function division. In actual implementation, there may be another division manner. For example, a plurality of units or components may be combined or integrated into another system, or some features may be ignored or not executed. In addition, the mutual coupling or direct coupling or communication connection shown or discussed may be indirect coupling or communication connection through some interfaces, apparatuses or units, and may be in electrical, mechanical or other forms.

The foregoing embodiments are only used to illustrate the technical solutions of the present application, rather than constitute a limitation thereto. Although the present application is described in detail with reference to the foregoing embodiments, it should be understood by a person of ordinary skill in the art that he/she may still modify the technical solutions described in the foregoing embodiments or equivalently replace some technical features therein; and these modifications or replacements do not make the essence of the corresponding technical solutions depart from the spirit and scope of the technical solutions of various embodiments of the present application.

What is claimed is:

1. An incidence rate monitoring method based on historical disease information, wherein
the incidence rate monitoring method based on the historical disease information comprises the following steps:
obtaining historical medical record data of a disease, and classifying the historical medical record data based on different pre-formed age ranges to obtain classified historical medical record data;
performing, based on the classified historical medical record data, an autonomous learning operation of a model training on the historical medical record data in each age range through a preset gated recurrent neural network and an ensemble learning algorithm to generate a prediction model, wherein the prediction model is configured to predict and calculate an incidence rate of a to-be-predicted disease; and
obtaining a type of the to-be-predicted disease, a to-be-predicted time point and related data before the to-be-predicted time point, inputting the related data into the prediction model, and calculating a predicted result of the incidence rate of the to-be-predicted disease at the to-be-predicted time point, wherein the related data comprises case data monitored before the to-be-predicted time point.

2. The incidence rate monitoring method based on the historical disease information according to claim 1, further comprising: extracting at least two training samples from the classified historical medical record data of each category through a random sample extraction;
selecting one training sample from the at least two training samples as an initial sample, and performing a preliminary model training based on the initial sample to obtain a model prototype of the prediction model; and
adding an information storage gate to the model prototype through the preset gated recurrent neural network, and using the at least two training samples extracted from each category to perform a secondary deep ensemble learning training on the model prototype with the information storage gate by using the ensemble learning algorithm, so as to construct the prediction model.

3. The incidence rate monitoring method based on the historical disease information according to claim 2, wherein the step of using the at least two training samples extracted from each category to perform the deep ensemble learning training on the model prototype with the information storage gate by using the ensemble learning algorithm, so as to construct the prediction model comprises:
performing a feature splitting training on each of the at least two training samples based on the ensemble learning algorithm to obtain first training features; and
sequentially inputting the first training features into the model prototype fora deep feature training to obtain a decision tree model with multiple branches, and using the decision tree model as the prediction model.

4. The incidence rate monitoring method based on the historical disease information according to claim 3, wherein before the step of obtaining the related data before the to-be-predicted time point, the incidence rate monitoring method further comprises:
obtaining medical ecological information corresponding to the historical medical record data, wherein the medical ecological information comprises at least one of weather data, medical level data and disease monitoring data; and
after the step of sequentially inputting the first training features into the model prototype for the deep feature training to obtain the decision tree model with the multiple branches, the incidence rate monitoring method further comprises:
performing a feature decomposition training on the medical ecological information by using the ensemble learning algorithm to obtain a second training feature; and
inputting the second training feature into the decision tree model for a tertiary deep training learning to construct a complete prediction model.

5. The incidence rate monitoring method based on the historical disease information according to claim 1, wherein
after the step of performing, based on the classified historical medical record data, the autonomous learning operation of the model training on the historical medical record data in the each age range through the preset gated recurrent neural network and the ensemble learning algorithm to generate the prediction model, the incidence rate monitoring method further comprises:
randomly capturing medical record data of a time period from the historical medical record data, and inputting the medical record data into the prediction model to obtain a predicted value of a number of cases corresponding to the medical record data of the time period;
determining whether the predicted value meets actual incidence data corresponding to the medical record data of the time period to obtain a model verification result; and
determining, based on the model verification result, whether to perform a quaternary deep training to optimize the prediction model, wherein the quaternary deep training is a process of repeating a secondary deep training and a tertiary deep training learning.

6. The incidence rate monitoring method based on the historical disease information according to claim 5, wherein after the step of obtaining the type of the to-be-predicted disease, the to-be-predicted time point and the related data before the to-be-predicted time point, inputting the related data into the prediction model, and calculating the predicted result of the incidence rate of the to-be-predicted disease at the to-be-predicted time point, the incidence rate monitoring method further comprises:
if it is determined that the model verification result is that the predicted value does not meet the actual incidence data, extracting N pieces of sample data from the historical medical record data, using an addition mechanism to update and/or reset the at least two training samples configured to train the prediction model to obtain updated and/or reset training samples, and training the prediction model based on the updated and/or reset training samples, wherein the N is greater than or equal to 2.

7. The incidence rate monitoring method based on the historical disease information according to claim 6, wherein the ensemble learning algorithm is a random forest algorithm.

8. An incidence rate monitoring device based on historical disease information, comprising a memory, a processor, and a computer program stored in the memory and executable on the processor, wherein the processor implements the following steps when executing the computer program:
obtaining historical medical record data of a disease, and classifying the historical medical record data based on different pre-formed age ranges to obtain classified historical medical record data;
performing, based on the classified historical medical record data, an autonomous learning operation of a model training on the historical medical record data in each age range through a preset gated recurrent neural network and an ensemble learning algorithm to generate a prediction model, wherein the prediction model is configured to predict and calculate an incidence rate of a to-be-predicted disease; and
obtaining a type of the to-be-predicted disease, a to-be-predicted time point and a related data before the to-be-predicted time point, inputting the related data into the prediction model, and calculating a predicted result of the incidence rate of the to-be-predicted disease at the to-be-predicted time point, wherein the related data comprises case data monitored before the to-be-predicted time point.

9. The incidence rate monitoring device based on the historical disease information according to claim 8, wherein the processor further implements the following steps when executing the computer program:
extracting at least two training samples from the classified historical medical record data of each category through a random sample extraction;
selecting one training sample from the at least two training samples as an initial sample, and performing a preliminary model training based on the initial sample to obtain a model prototype of the prediction model; and
adding an information storage gate to the model prototype through the preset gated recurrent neural network, and using the at least two training samples extracted from each category to perform a secondary deep ensemble learning training on the model prototype with the information storage gate by using the ensemble learning algorithm, so as to construct the prediction model.

10. The incidence rate monitoring device based on the historical disease information according to claim 9, wherein the processor further implements the following steps when executing the computer program:
performing a feature splitting training on each of the at least two training samples based on the ensemble learning algorithm to obtain first training features; and
sequentially inputting the first training features into the model prototype fora deep feature training to obtain a decision tree model with multiple branches, and using the decision tree model as the prediction model.

11. The incidence rate monitoring device based on the historical disease information according to claim 10, wherein the processor further implements the following steps when executing the computer program:
obtaining medical ecological information corresponding to the historical medical record data, wherein the medical ecological information comprises at least one of weather data, medical level data and disease monitoring data; and
after the step of sequentially inputting the first training features into the model prototype for the deep feature training to obtain the decision tree model with the multiple branches, the incidence rate monitoring method further comprises:
performing a feature decomposition training on the medical ecological information by using the ensemble learning algorithm to obtain a second training feature; and
inputting the second training feature into the decision tree model for a tertiary deep training learning to construct a complete prediction model.

12. The incidence rate monitoring device based on the historical disease information according to claim 8, wherein the processor further implements the following steps when executing the computer program:
randomly capturing medical record data of a time period from the historical medical record data, and inputting the medical record data into the prediction model to obtain a predicted value of a number of cases corresponding to the medical record data of the time period;
determining whether the predicted value meets actual incidence data corresponding to the medical record data of the time period to obtain a model verification result; and
determining, based on the model verification result, whether to perform a quaternary deep training to optimize the prediction model, wherein the quaternary deep training is a process of repeating the secondary deep training and the tertiary deep training learning.

13. The incidence rate monitoring device based on the historical disease information according to claim 12, wherein the processor further implements the following steps when executing the computer program:
if it is determined that the model verification result is that the predicted value does not meet the actual incidence data, extracting N pieces of sample data from the historical medical record data, using an addition mechanism to update and/or reset the at least two training samples configured to train the prediction model to obtain updated and/or reset training samples, and training the prediction model based on the updated and/or reset training samples, wherein the N is greater than or equal to 2.

14. The incidence rate monitoring device based on the historical disease information according to claim 13, wherein the processor further implements the following steps when executing the computer program:
the ensemble learning algorithm is a random forest algorithm.

15. A computer-readable storage medium, wherein a computer-readable storage medium stores computer instructions, and when the computer instructions are run on a computer, the computer is enabled to perform the following steps:
obtaining historical medical record data of a disease, and classifying the historical medical record data based on different pre-formed age ranges to obtain classified historical medical record data;
performing, based on the classified historical medical record data, an autonomous learning operation of a model training on the historical medical record data in each age range through a preset gated recurrent neural network and an ensemble learning algorithm to generate a prediction model, wherein the prediction model is configured to predict and calculate an incidence rate of a to-be-predicted disease; and
obtaining a type of the to-be-predicted disease, a to-be-predicted time point, and a related data before the to-be-predicted time point, inputting the related data into the prediction model, and calculating a predicted result of the incidence rate of the to-be-predicted disease at the to-be-predicted time point, wherein the related data comprises case data monitored before the to-be-predicted time point.

16. The computer-readable storage medium according to claim 15, wherein when the computer instructions are run on the computer, the computer is further enabled to perform the following steps:
extracting at least two training samples from the classified historical medical record data of each category through a random sample extraction;
selecting one training sample from the at least two training samples as an initial sample, and performing a preliminary model training based on the initial sample to obtain a model prototype of the prediction model; and
adding an information storage gate to the model prototype through the preset gated recurrent neural network, and using the at least two training samples extracted from each category to perform a secondary deep ensemble learning training on the model prototype with the information storage gate by using the ensemble learning algorithm, so as to construct the prediction model.

17. The computer-readable storage medium according to claim 16, wherein when the computer instructions are run on the computer, the computer is further enabled to perform the following steps:
performing a feature splitting training on each of the at least two training samples based on the ensemble learning algorithm to obtain first training features; and
sequentially inputting the first training features into the model prototype fora deep feature training to obtain a decision tree model with multiple branches, and using the decision tree model as the prediction model.

18. The computer-readable storage medium according to claim 17, wherein when the computer instructions are run on the computer, the computer is further enabled to perform the following steps:
obtaining medical ecological information corresponding to the historical medical record data, wherein the medical ecological information comprises at least one of weather data, medical level data and disease monitoring data; and
after the step of sequentially inputting the first training features into the model prototype for the deep feature training to obtain the decision tree model with the multiple branches, the incidence rate monitoring method further comprises:
performing a feature decomposition training on the medical ecological information by using the ensemble learning algorithm to obtain a second training feature; and
inputting the second training feature into the decision tree model for a tertiary deep training learning to construct a complete prediction model.

19. The computer-readable storage medium according to claim 15, wherein when the computer instructions are run on the computer, the computer is further enabled to perform the following steps:
randomly capturing medical record data of a time period from the historical medical record data, and inputting the medical record data into the prediction model to obtain a predicted value of a number of cases corresponding to the medical record data of the time period;
determining whether the predicted value meets actual incidence data corresponding to the medical record data of the time period to obtain a model verification result; and
determining, based on the model verification result, whether to perform a quaternary deep training to optimize the prediction model, wherein the quaternary deep training is a process of repeating the secondary deep training and the tertiary deep training learning.

20. (canceled)

21. The incidence rate monitoring method based on the historical disease information according to claim 5, further comprising: extracting at least two training samples from the classified historical medical record data of each category through a random sample extraction;
selecting one training sample from the at least two training samples as an initial sample, and performing a preliminary model training based on the initial sample to obtain a model prototype of the prediction model; and
adding an information storage gate to the model prototype through the preset gated recurrent neural network, and using the at least two training samples extracted from each category to perform the secondary deep ensemble learning training on the model prototype with the information storage gate by using the ensemble learning algorithm, so as to construct the prediction model.
